(19) United States
(12) Patent Application Publication
(10) Pub. No.: US 2006/0089352 A1
(43) Pub. Date: Apr. 27, 2006

(54) Substituted thiazines as material protecting agents

(76) Inventors: Rainer Bruns, Leverkusen (DE); Oliver Kretschik, Koln (DE); Hermann Uhr, Leverkusen (DE); Martin Kugler, Leichlingen (DE); Peter Wachtler, Krefeld (DE)

Correspondence Address: Law & Intellectual Property Department;Lanxess Corporation, 100 Bayer Road, Pittsburgh, PA, 15275-1112, US

(21) Appl. No.: 10/522,865

(22) PCT Filed: Jul. 14, 2003
(86) PCT No.: PCT/EP03/07587

§ 371 Date: Sep. 19, 2005

(30) Foreign Application Priority Data
Jul. 29, 2002 (DE) 102 34 425.6

(51) Int. Cl. C07D 279/06; A01N 43/86

(52) U.S. Cl. 514/227.2; 544/53

(57) ABSTRACT

The invention relates to novel thiazines of formula (I), wherein the substituents R¹ to R⁷ have the designation cited in the description, and to the metallic salts and acid addition compounds thereof. Said novel thiazines have an excellent capacity for protecting technical materials from being infected by micro-organisms.

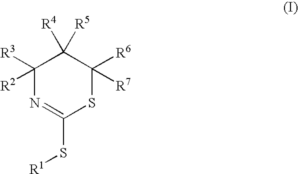

The present invention relates to novel thiazines, to processes for their preparation and to their use for controlling unwanted microorganisms, to novel mixtures of thiazines with other active compounds and also to novel intermediates and processes for their preparation.

From the literature, it is known that some unsubstituted thiazines can be used as agricultural and horticultural fungicides (cf. JP-A 2000-119263). The use of thiazines as biocides for protecting industrial materials has hitherto not been described.

Surprisingly, it has now been found that the thiazines of the formula (I) according to the invention have better fungicidal activities than the prior-art compounds of the most similar constitution. Furthermore, it has been found that the novel thiazines of the formula (I) are highly suitable for protecting industrial materials against attack by microorganisms.

The present invention provides thiazines of the formula (I) 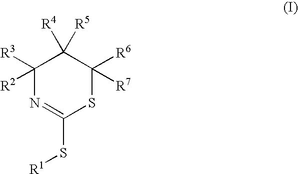
in which
R¹ represents hydrogen or represents in each case optionally substituted alkyl, aryl or heterocyclyl,
and
R² to R⁷ independently of one another represent hydrogen or represent in each case optionally substituted alkyl, aryl or cycloalkyl, where at least one of the substituents R² to R⁷ is different from hydrogen,
or
in each case two substituents from the group consisting of R² to R⁷, together with the C atoms to which they are attached, represent an optionally substituted carbocyclic ring, and the remaining substituents from the group consisting of R² to R⁷ represent hydrogen,
and their metal salts and acid addition compounds.

In the context of the present invention, the alkyl radicals mentioned are straight-chain or branched, unsubstituted or substituted and contain 1 to 12 C atoms, in particular 1 to 8 C atoms. Preferred alkyl radicals are methyl, ethyl, butyl and octyl. Cycloalkyl generally represents an unsubstituted or substituted cycloalkyl radical having 3 to 8 C atoms, in particular 3 to 7 C atoms. Preference is given to cyclopropyl and cyclohexyl. Aryl generally represents a substituted or unsubstituted aromatic radical, in particular phenyl and naphthyl. Halogen generally represents fluorine, chlorine, bromine or iodine, in particular fluorine, chlorine and bromine. Heterocyclyl represents a saturated, unsaturated or aromatic, substituted or unsubstituted 5- to 7-membered ring, in particular 5- or 6-membered ring, having one or more identical or different heteroatoms, in particular 1 to 4 heteroatoms and preferably 1 to 3 heteroatoms. Heteroatoms are in particular N, O and S, preferably N and S. If appropriate, a further carbocyclic ring, in particular a 6-membered carbocyclic ring, preferably a 6-membered aromatic ring, may be fused onto the heterocyclyl radical. The term carbocyclic ring denotes a saturated or mono- to polyunsaturated, substituted or unsubstituted carbocyclic ring having 3 to 12 C atoms, in particular 3 to 8 C atoms.

The radicals mentioned above are optionally mono- to polysubstituted by identical or different substituents, in particular mono- to pentasubstituted, preferably mono- or trisubstituted, with the substituents being in each case: halogen, in particular fluorine, chlorine, bromine; alkyl, in particular C₁-C₈-alkyl; cycloalkyl, in particular C₃-C₈-cycloalkyl; haloalkyl, in particular C₁-C₆-haloalkyl; alkoxy, in particular C₁-C₆-alkoxy; haloalkoxy, in particular C₁-C₆-haloalkoxy; nitro; nitrilo; amino; alkylamino or dialkylamino (together referred to as (di)alkylamino, in particular (di)-C₁-C₆-alkylamino; hydroxyl; phenyl, biphenyl; naphthyl; phenoxy and phenoxyphenyl.

Preference is given to compounds of the formula (I) in which
R¹ represents hydrogen or represents in each case optionally substituted C₁-C₁₂-alkyl, C₆-C₁₀-aryl or 5- to 7-membered heterocyclyl having 1 to 4 identical or different heteroatoms, which optionally contains a fused-on 6-membered aromatic ring,
and
R² to R⁷ independently of one another represent hydrogen or represent in each case optionally substituted C₁-C₁₂-alkyl, C₆-C₁₀-aryl or C₃-C₈-cycloalkyl, where at least one of the substituents R² to R⁷ is different from hydrogen,
or
two substituents from the group consisting of R² to R⁷, together with the C atoms, to which they are attached, represent an optionally substituted 3- to 12-membered carbocyclic ring and the remaining substituents from the group consisting of R² to R⁷ represent hydrogen.

Particular preference is given to compounds of the formula (I) in which
R¹ represents hydrogen, represents C₁-C₈-alkyl which is optionally mono- to pentasubstituted by identical or different substituents from the group consisting of halogen, C₁-C₆-alkoxy, C₁-C₆-haloalkyl and C₁-C₆-haloalkoxy, represents C₆-C₁₀-aryl which is mono- to trisubstituted by identical or different substituents from the group consisting of halogen, C₁-C₈-alkyl, C₁-C₆-alkoxy, C₁-C₆-haloalkyl, C₁-C₆-haloalkoxy, hydroxyl, nitro, nitrilo, amino, (di)-C₁-C₆-alkylamino, C₃-C₈-cycloalkyl, phenyl and phenoxy, or represents 5- to 6-membered heterocyclyl having 1 to 3 identical or different heteroatoms from the group consisting of N, O, S, which optionally contains a fused-on aromatic 6-membered ring and which is optionally substituted by C₁-C₈-alkyl,
and
R² to R⁷ independently of one another represent hydrogen, represent C₁-C₁₂-alkyl which is optionally mono- to pentasubstituted by identical or different substituents from the group consisting of halogen, C₁-C₆-alkoxy, C₁-C₆-haloalkyl and C₁-C₆-haloalkoxy, represent C₆-C₁₀-aryl which is optionally mono- to pentasubstituted by identical or different substituents from the group consisting of halogen, C₁-C₈-alkyl, C₁-C₆-alkoxy, C₁-C₆-haloalkyl and C₁-C₆-haloalkoxy, or represent C₃-C₈-cycloalkyl which is optionally mono- to pentasubstituted by identical or different substituents from the group consisting of halogen, C₁-C₆-alkoxy, C₁-C₆-haloalkyl and C₁-C₆-haloalkoxy, where at least one of the substituents R² to R⁷ is different from hydrogen,
or
two substituents from the group consisting of R² to R⁷, together with the C atoms to which they are attached, represent a 3- to 12-membered carbocyclic ring which is optionally mono- to pentasubstituted by identical or different substituents from the group consisting of halogen, C₁-C₈-alkyl, C₁-C₆-alkoxy, C₁-C₆-haloalkyl and C₁-C₆-haloalkoxy, and the remaining substituents from the group consisting of R² to R⁷ represent hydrogen.

Very particular preference is given to compounds of the formula (I) in which
R¹ represents hydrogen, represents C₁-C₈-alkyl which is optionally mono- to trisubstituted by identical or different substituents from the group consisting of fluorine, chlorine, bromine, C₁-C₃-alkoxy, C₁-C₂-haloalkyl and C₁-C₂-haloalkoxy or represents phenyl or naphthyl, each of which is mono- to trisubstituted by identical or different substituents from the group consisting of fluorine, chlorine, bromine, C₁-C₈-alkyl, C₁-C₃-alkoxy, C₁-C₂-haloalkyl, C₁-C₂-haloalkoxy, hydroxyl, nitro, nitrilo, amino, (di)-C₁-C₃-alkylamino, C₃-C₈-cycloalkyl, phenyl and phenoxy, or represents 5- or 6-membered heterocyclyl having 1 or 2 identical or different heteroatoms from the group consisting of N, O, S, which optionally contains a fused-on aromatic 6-membered ring and which is optionally substituted by C₁-C₄-alkyl,
and
R² to R⁷ independently of one another represent hydrogen, represent C₁-C₈-alkyl which is optionally mono- to trisubstituted by identical or different substituents from the group consisting of fluorine, chlorine, bromine, C₁-C₃-alkoxy, C₁-C₂-haloalkyl and C₁-C₂-haloalkoxy, represent phenyl which is optionally mono- to trisubstituted by identical or different substituents from the group consisting of fluorine, chlorine, bromine, C₁-C₄-alkyl, C₁-C₄-alkoxy C₁-C₂-haloalkyl and C₁-C₂-haloalkoxy or represent C₃-C₇-cycloalkyl which is optionally mono- to trisubstituted by identical or different substituents from the group consisting of fluorine, chlorine, bromine, C₁-C₄-alkyl, C₁-C₄-alkoxy C₁-C₂-haloalkyl and C₁-C₂-haloalkoxy, where at least one of the substituents R² to R⁷ is different from hydrogen,
or
two substituents from the group consisting of R² to R⁷, together with the C atoms, to which they are attached, represent the 3- to 8-membered carbocyclic ring which is optionally mono- to trisubstituted by identical or different substituents from the group consisting of fluorine, chlorine, bromine, C₁-C₄-alkyl, C₁-C₄-alkoxy C₁-C₂-haloalkyl and C₁-C₂-haloalkoxy, and the remaining substituents from the group consisting of R² to R⁷ represent hydrogen.

Especially preferred are the compounds of the formula (I) a) to j) where in each case R¹ has the general and preferred meanings given above: 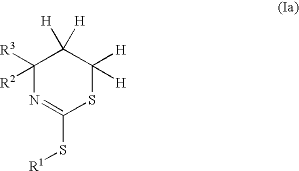
where
R² and R³ independently of one another represent optionally substituted alkyl, preferably C₁-C₁₂-alkyl and in particular C₁-C₈-alkyl; 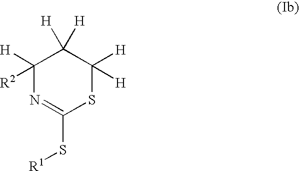
where
R² represents optionally substituted alkyl, cycloalkyl or aryl, preferably C₁-C₁₂-alkyl, C₃-C₈-cycloalkyl or phenyl which is optionally mono- or polysubstituted by identical or different substituents from the group consisting of halogen, C₁-C₈-alkyl and C₁-C₆-alkoxy, and represents in particular C₁-C₈-alkyl, C₃-C₇-cycloalkyl or phenyl which is optionally mono- to trisubstituted by identical or different substituents from the group consisting of chlorine, bromine, fluorine, C₁-C₄-alkyl and C₁-C₄-alkoxy; 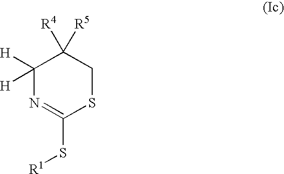
where
R⁴ represents optionally substituted alkyl and R⁵ represents hydrogen or optionally substituted alkyl, preferably, R⁴ represents C₁-C₁₂-alkyl and R⁵ represents hydrogen or C₁-C₁₂-alkyl, and in particular, R⁴ represents C₁-C₈-alkyl and R⁵ represents hydrogen or C₁-C₈-alkyl; 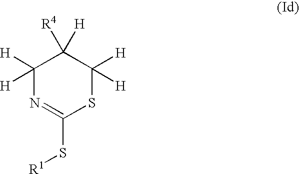
where
R⁴ represents optionally substituted aryl or cycloalkyl, preferably represents C₃-C₈-cycloalkyl or phenyl which is optionally mono- or polysubstituted by identical or different substituents from the group consisting of halogen, C₁-C₈-alkyl and C₁-C₆-alkoxy, and in particular represents C₃-C₇-cycloalkyl or phenyl which is optionally mono- to trisubstituted by identical or different substituents from the group consisting of chlorine, bromine, fluorine, C₁-C₈-alkyl, C₁-C₃-alkoxy; 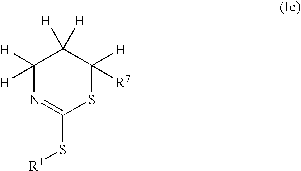
where
R⁷ represents optionally substituted alkyl, aryl or cycloalkyl, preferably represents C₁-C₁₂-alkyl, C₃-C₈-cycloalkyl or phenyl which is optionally mono- or polysubstituted by identical or different substituents from the group consisting of halogen, C₁-C₈-alkyl and C₁-C₆-alkoxy, and in particular represents C₁-C₈-alkyl, C₃-C₇-cycloalkyl or phenyl which is optionally mono- to trisubstituted by identical or different substituents from the group consisting of chlorine, bromine, fluorine, C₁-C₈-alkyl, C₁-C₃-alkoxy; 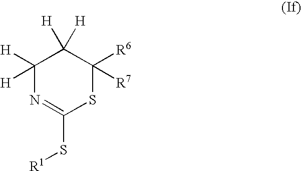
where
R⁶ and R⁷ independently of one another represent optionally substituted alkyl, preferably represent C₁-C₁₂-alkyl and in particular represent C₁-C₈-alkyl; 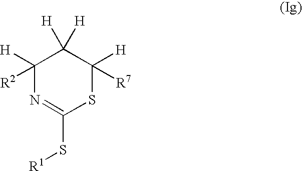
where
R² and R⁷ independently of one another represent optionally substituted alkyl, preferably represent C₁-C₁₂-alkyl and in particular represent C₁-C₈-alkyl; 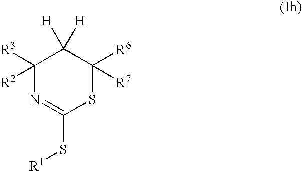
where
R², R³ and R⁷ independently of one another represent optionally substituted alkyl and R⁶ represents hydrogen or optionally substituted alkyl, preferably, R², R³ and R⁷ represent C₁-C₁₂-alkyl and R⁶ represents hydrogen or C₁-C₁₂-alkyl, and in particular, R², R³ and R⁷ represent C₁-C₈-alkyl and R⁶ represents hydrogen or C₁-C₈-alkyl; 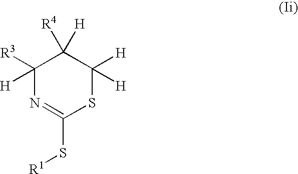
where
R³ and R⁴ together with the C atoms to which they are attached represent an optionally substituted carbocyclic ring, preferably represent a 3- to 12-membered carbocyclic ring and in particular represent a 3- to 8-membered carbocyclic ring; 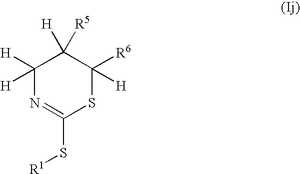
where
R⁵ and R⁶ together with the C atoms to which they are attached represent an optionally substituted carbocyclic ring, preferably represent a 3- to 12-membered carbocyclic ring and in particular represent a 3- to 8-membered carbocyclic ring.

Some of the compounds of the general formula (I) can be present as cis or transisomers. The present invention provides both the isomer mixtures and the compounds which are isomerically pure or isomerically enriched by chromatographic methods.

The novel compounds of the formula (I) can be prepared by reacting mercaptans of the formula (II) or salts thereof 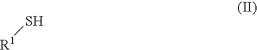
in which
R¹ is as defined above
with compounds of the formula (III) 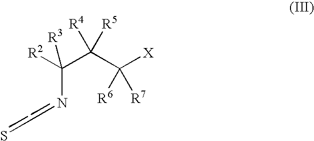
in which
R² to R⁷ are as defined above,
and represents X halogen or a leaving group,
if appropriate in the presence of a diluent and if appropriate in the presence of an acid binder and if appropriate in the presence of a catalyst.

Alternatively, the novel compounds of the formula (I) can be prepared by
a) diazotizing primary amines of the formula (IV) 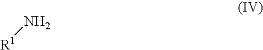
in which
R¹ is as defined above
with a diazotizing agent and reacting them with compounds of the formula (V) or salts thereof 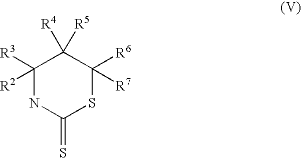
in which
R² to R⁷ are as defined above,
if appropriate in the presence of a diluent and if appropriate in the presence of a catalyst, or
b) reacting compounds of the formula (VI) 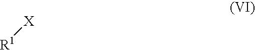
in which
R¹ is as defined above,
and
X represents halogen or a leaving group
with compounds of the formula (V) or salts thereof 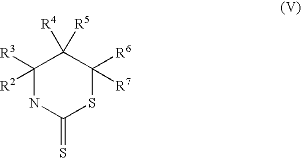
in which
R² to R⁷ are as defined above,
if appropriate in the presence of a diluent and if appropriate in the presence of an acid binder.

Except for
4-methyl-1,3-thiazinane-2-thione, CAS 5554-49-4;
4,6,6-trimethyl-1,3-thiazinane-2-thione, CAS 6268-74-2;
6-methyl-1,3-thiazinane-2-thione, CAS 13091-77-5;
5-methyl-4-phenyl-1,3-thiazinane-2-thione, CAS 37814-88-3;
4,4,6-trimethyl-1,3-thiazinane-2-thione, CAS 79696-63-2,

the intermediates of the formula (V) are novel and also form part of the subject-matter of the present invention.

The present invention also provides the preparation of the novel intermediates of the formula (V) by reacting compounds of the formula (VII) or salts thereof 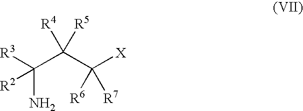
in which
R² to R⁷ are as defined above,
and
X represents halogen or a leaving group
with carbon sulfide, if appropriate in the presence of a diluent and if appropriate in the presence of an acid binder.

The salts can either be prepared and reacted in situ or used neat. Suitable salts are in particular the alkali metal and alkaline earth metal salts, preferably the alkali metal salts and particularly preferably the sodium and potassium salts. The preparation of the salts is carried out by customary chemical methods.

The starting materials of the formulae (II), (IV) and (VI) are commercially available, described in the literature or preparable by simple chemical operations.

If appropriate, the compounds of formulae (III) and (V) and (VII) can be generated in situ and reacted directly, or they can be employed as a pure substance.

Suitable diluents, which are added, if appropriate, include both water and any customary inert organic solvents. These preferably include hydrocarbons, such as toluene, xylene or hexane, chlorinated hydrocarbons, such as chlorobenzene, methylene chloride or chloroform, ketones, such as acetone or butanone, ethers, such as tetrahydrofuran, diethyl ether, methyl tert-butyl ether, dimethoxyethane or dioxane, nitriles, such as acetonitrile, amides, such as N,N-dimethylformamide, N,N-dimethylacetamide or N-methylpyrrolidone, sulfoxides, such as dimethyl sulfoxide, sulfones, such as sulfolane, and also esters, such as ethyl acetate or methyl acetate.

In the preparation process, the reaction temperatures can be varied within a wide temperature range. In general, the processes are carried out between −30° C. and +150° C., preferably between 0° C. and +110° C.

When carrying out the process according to the invention, in general from 1 to 10 mol, preferably from 1 to 5 mol, of the compounds of the formula (II) or (IV) or (VI) are employed per mole of the starting material of the formula (III) or (V) or (VII). Work-up is carried out by customary methods.

Suitable for use as acid binders are both organic and inorganic bases. Suitable inorganic bases are carbonates, hydroxides, phosphates and hydrides of the alkali metals, alkaline earth metals and transition metals; preference is given to using the carbonates, hydroxides and hydrides of the alkali metals and alkaline earth metals. Particularly preferred are potassium carbonate, sodium carbonate, cesium carbonate, sodium hydroxide, potassium hydroxide and also sodium hydride and potassium hydride. Suitable for use as organic bases are primary, secondary and tertiary amines. Preference is given to tertiary amines, such as trimethylamine, triethylamine, tributylamine, DBU, DBN and also pyridine, N,N-dimethylaniline or N,N-dimethylpyridine.

The diazotizations can be carried out in the presence of an alkali metal nitrite or an alkyl nitrite. Suitable for use as alkali metal nitrite are all customary alkali metal nitrites; preference is given to using sodium nitrite or potassium nitrite. Suitable for use as alkyl nitrite are all customary alkyl nitrites, preferably those having 1 to 10 carbon atoms, in particular methyl nitrite, ethyl nitrite, n-propyl nitrite, i-propyl nitrite and isoamyl nitrite. Suitable for use as diazotization catalysts are copper, copper salts, palladium or palladium salts. Preference is given to copper turnings, copper(I) iodide, palladium(II) acetate or tetrakis(triphenylphoshine)palladium(0).

The process according to the invention is generally carried out under atmospheric pressure. However, it is also possible to operate under reduced or elevated pressures, i.e. in the range from 0.1 to 10 bar.

Suitable for use as leaving groups are the esters of the sulfonic acids, in particular mesylates, tosylates or triflates.

Suitable for use as catalysts are, if appropriate, Lewis acids, in particular Lewis acids which are soft according to the HSAB concept, or N,N-dimethylaminopyridine.

The present invention also provides the metal salts and acid addition compounds of the compounds of the formula (I).

Preferred metal salts are salts of metals of the II. to IV. main group and the I. and II. and the IV. to VII. transition group of the Periodic Table of the Elements, examples which may be mentioned being copper, zinc, manganese, magnesium, tin, iron, calcium, aluminum, lead, chromium, cobalt and nickel.

Suitable anions of the salts are those which can preferably be derived from the following acids: hydrohalic acids, such as, for example, hydrochloric acid and hydrobromic acid, furthermore phosphoric acid, nitric acid and sulfuric acid.

The metal salt complexes of the compounds of the formula (I) can in a simple manner by customary processes, for example by dissolving the metal salts in alcohol, for example ethanol, and adding the solution to compounds of the formula (I). The metal salt complexes can be isolated in a known manner, for example by filtration, and, if appropriate, be purified by recrystallization.

Acids suitable for preparing the acid addition compounds of the compounds of the formula (I) are preferably the following acids: the hydrohalic acids, for example hydrochloric acid and hydrobromic acid, in particular hydrochloric acid, furthermore phosphoric acid, nitric acid, sulfuric acid, mono- and bifunctional carboxylic acids and hydroxycarboxylic acids, such as, for example, acetic acid, propionic acid, 2-ethylhexanoic acid, butyric acid, mandelic acid, oxalic acid, succinic acid, 2-hydroxyethanedicarboxylic acid, maleic acid, fumaric acid, tartaric acid, citric acid, salicylic acid, sorbic acid, lactic acid, and also sulfonic acids, such as, for example, p-toluenesulfonic acid, 1,5-naphthalenedisulfonic acid, alkanesulfonic acids, benzoic acid and optionally substituted benzoic acids.

The acid addition salts of the compounds of the formula (I) can be obtained in a simple manner by customary methods forming salts, for example by dissolving a compound of the formula (I) in a suitable inert solvent and adding the acid, for example hydrochloric acid, and be isolated in a known manner, for example by filtration, and, if appropriate, be purified by washing with an inert organic solvent.

Compounds of the formula (I) have strong microbicidal action and can be used for controlling unwanted microorganisms, such as, for example, fungi, bacteria and algae. The compounds of the formula (I) are preferably used for controlling unwanted microorganisms in the protection of materials.

In the protection of materials, the compounds according to the invention can be used for protecting industrial materials against attack and destruction by unwanted microorganisms.

In the present context, industrial materials are to be understood as meaning non-living materials which have been prepared for use in industry. Industrial materials are, for example, glues, sizes, paper and board, textiles, leather, wood, timber products, paints and synthetic articles, cooling lubricants and other materials which can be attacked or destroyed by microorganisms. Parts of production plants, for example cooling-water circuits, which may be impaired by the multiplication of microorganisms may also be mentioned as industrial materials in the context of the present invention. Industrial materials which are preferably to be protected are glues, sizes, paper and board, leather, wood, paints, synthetic articles, cooling lubricants and heat transfer liquids.

The compounds of the formula (I) according to the invention are particularly suitable for protecting wood, plastics, cooling lubricants and coating systems, such as paints, varnishes or plasters against attack by microorganisms. The compounds of the formula (I) according to the invention are very preferably suitable for protecting wood, plastics and coating systems, such as paints, varnishes or plasters against attack by microorganisms.

Examples of microorganisms which are capable of brining about degradation of, or change in, the industrial materials and which may be mentioned are bacteria, fungi, yeast, algae and slime organisms. The active compounds of the formula (I) according to the invention preferably act against fungi, in particular molds, wood-discoloring and wood-destroying fungi (Basidiomycetes) and also against slime organisms and bacteria. Particularly preferred is the action of the active compounds of the formula (I) as film fungicides.

Microorganisms of the following genera may be mentioned by way of example:
Alternuria, such as Alternaria tenuis,
Aspergillus, such as Aspergillus niger,
Chaetomium, such as Chaetomium globosum,
Coniophora, such as Coniophora puetana,
Lentinus, such as Lentinus tigrinus,
Penicillium, such as Penicillium glaucum,
Polyporus, such as Polyporus versicolor,
Aureobasidium, such as Aureobasidium pullulans,
Sclerophoma, such as Sclerophoma pityophila,
Trichoderma, such as Trichoderma viride,
Escherichia, such as Escherichia coli,
Pseudomonas, such as Pseudomonas aeruginosa,
Staphylococcus, such as Staphylococcus aureus.

Depending on their respective physical and/or chemical properties, the active compounds can be converted into customary formulations, such as solutions, emulsions, suspensions, powders, foams, pastes, granules, aerosols, very fine capsules in polymeric substances and in coating materials for seed, also HLV cold- and warm-fogging formulations.

These formulations and compositions are prepared in a known manner, for example by mixing the active compounds with extenders, that is, liquid solvents, liquefied gases under pressure, and/or solid carriers, if appropriate with the use of surfactants, that is emulsifiers and/or dispersants and/or foam-formers. If the extender used is water, it is also possible to use for example organic solvents as auxiliary solvents. Essentially, suitable liquid solvents are: aromatics, such as xylene, toluene or alkylnaphthalenes, chlorinated aromatics or chlorinated aliphatic hydrocarbons, such as chlorobenzenes, chloroethylene or methylene chloride, aliphatic hydrocarbons, such as cyclohexane or paraffins, for example mineral oil fractions, alcohols, such as butanol or glycol and their ethers and esters, ketones, such as acetone, methyl ethyl ketone, methyl isobutyl ketone or cyclohexanone, strongly polar solvents, such as dimethyl formamide and dimethyl sulfoxide, and water. By liquefied gaseous extenders or carriers are meant liquids which are gaseous at ambient temperature and under atmospheric pressure, for example aerosol propellants, such as halogenated hydrocarbons and butane, propane, nitrogen and carbon dioxide. Suitable solid carriers are: for example ground natural minerals, such as kaolins, clays, talc, chalk, quartz, attapulgite, montmorillonite or diatomaceous earth, and ground synthetic minerals, such as finely divided silica, alumina and silicates. Suitable solid carriers for granules are: for example crushed and fractionated natural rocks such as calcite, marble, pumice, sepiolite and dolomite, and synthetic granules of organic and inorganic meals, and granules of organic material such as sawdust, coconut shells, maize hobs and tobacco stalks. Suitable emulsifiers and/or foam-formers are: for example nonionic and anionic emulsifiers, such as polyoxyethylene fatty acid esters, polyoxyethylene fatty alcohol ethers, for example alkylaryl polyglycol ethers, alkylsulfonates, alkyl sulfates, arylsulfonates and protein hydrolysates. Suitable dispersants are: for example ligninosulfite waste liquors and methylcellulose.

Tackifiers such as carboxymethylcellulose and natural and synthetic polymers in the form of powders, granules or latices, such as gum arabic, polyvinyl alcohol and polyvinyl acetate, and natural phospholipids, such as cephalins and lecithins, and synthetic phospholipids, can be used in the formulations. Other possible additives are mineral and vegetable oils.

It is possible to use colorants such as inorganic pigments, for example iron oxide, titanium oxide and Prussian blue, and organic dyestuffs, such as alizarin dyestuffs, azo dyestuffs and metal phthalocyanin dyestuffs, and trace nutrients such as salts of iron, manganese, boron, copper, cobalt, molybdenum and zinc.

The formulations generally comprise between 0.1 and 95% by weight of active compound, preferably between 0.5 and 90% by weight.

The active compounds according to the invention can be used as such or in their formulations, also in mixtures with known fungicides, bactericides, acaricides, nematicides or insecticides, for example to broaden the activity spectrum or to prevent the development of resistance. In many cases, synergistic effects are obtained, i.e. the activity of the mixture is greater than the activity of the individual components.

The efficacy and the activity spectrum of the active compounds of the formula (I) and of the compositions preparable therefrom, of precursors or of formulations in general can be increased by adding, if appropriate, further antimicrobial compounds, fungicides, bactericides, herbicides, insecticides or other active compounds, so as to widen the spectrum of activity or to obtain particular effects such as, for example, additional protection against insects. These mixtures may have a wider activity spectrum than the compounds according to the invention.

In many cases, synergistic effects are obtained, i.e. the activity of the mixture is greater than the activity of the individual components. The following co-components are found to be particularly favorable:

Triazoles Such As:

azaconazole, azocyclotin, bitertanol, bromuconazole, cyproconazole, diclobutrazole, difenoconazole, diniconazole, epoxyconazole, etaconazole, fenbuconazole, fenchlorazole, fenethanil, fluquinconazole, flusilazole, flutriafol, furconazole, hexaconazole, imibenconazole, ipconazole, isozofos, myclobutanil, metconazole, paclobutrazol, penconazole, propioconazole, prothioconazole, simeoconazole, (±)-cis-1-(4-chlorophenyl)-2-(1H-1,2,4-triazol-1-yl)cycloheptanol, 2-(1-tert-butyl)-1-(2-chlorophenyl)-3-(1,2,4-triazol-1-yl)propan-2-ol, tebuconazole, tetraconazole, triadimefon, triadimenol, triapenthenol, triflumizole, triticonazole, uniconazole and their metal salts and acid adducts;

Imidazoles Such As:

clotrimazole, bifonazole, climbazole, econazole, fenapamil, imazalil, isoconazole, ketoconazole, lombazole, miconazole, pefurazoate, prochloraz, triflumizole, thiazolcar, 1-imidazolyl-1-(4′-chlorophenoxy)-3,3-dimethylbutan-2-one, and their metal salts and acid adducts;

Pyridines and Pyrimidines Such As:

ancymidol, buthiobate, fenarimol, mepanipyrin, nuarimol, pyvoxyfuir, triamirol;

Succinate Dehydrogenase Inhibitors Such As:

benodanil, carboxim, carboxim sulfoxide, cyclafluramid, fenfuram, flutanil, furcarbanil, furmecyclox, mebenil, mepronil, methfuroxam, metsulfovax, pyrocarbolid, nicobifen, oxycarboxin, Shirlan, Seedvax;

Naphthalene Derivatives Such As:

terbinafine, naftifine, butenafine, 3-chloro-7-(2-aza-2,7,7-trimethyloct-3-en-5-yne);

Sulfenamides Such As:

dichlofluanid, tolylfluanid, folpet, fluorofolpet, captan, captofol;

Benzimidazoles Such As:

carbendazim, benomyl, fuberidazole, thiabendazole or their salts;

Morpholine Derivatives Such As:

aldimorph, dimethomorph, dodemorph, falimorph, fenpropidin fenpropimorph, tridemorph, trimorphamid and their arylsulfonate salts such as, for example, p-toluenesulfonic acid and p-dodecylphenylsulfonic acid;

Benzothiazoles Such As:

2-mercaptobenzothiazole;

Benzothiophene Dioxides Such As:

N-cyclohexyl-benzo[b]thiophenecarboxamide S,S-dioxide;

Benzamides Such As:

2,6-dichloro-N-(4-trifluoromethylbenzyl)-benzamide, tecloftalam;

Boron Compounds Such As:

boric acid, boric ester, borax;

Formaldehyde and Formaldehyde-Releasing Compounds Such As:

benzyl alcohol mono-(poly)-hemiformal, n-butanol hemiformal, dazomet, ethylene glycol hemiformal, hexahydro-S-triazine, hexamethylenetetramine, N-hydroxymethyl-N′-methylthiourea, N-methylolchloroacetamide, oxazolidine, paraformaldehyde, taurolin, tetrahydro-1,3-oxazine, N-(2-hydroxypropyl)aminemethanol, tetramethyloylacetylenediurea;

Isothiazolinones Such As:

N-methylisothiazolin-3-one, 5-chloro-N-methylisothiazolin-3-one, 4,5-dichloro-N-octylisothiazolin-3-one, 5-chloro-N-octylisothiazolinone, N-octylisothiazolin-3-one, 4,5-trimethyleneisothiazolinone, 4,5-benzoisothiazolinone;

Aldehydes Such As:

cinnamaldehyde, formaldehyde, glutardialdehyde, β-bromocinnamaldehyde, o-phthaldialdehyde;

Thiocyanates Such As:

thiocyanatomethylthiobenzothiazole, methylenebisthiocyanate;

Quaternary Ammonium Compounds and Guanidine Such As:

benzalkonium chloride, benzyldimethyltetradecylammonium chloride, benzyldimethyldodecylammonium chloride, dichlorobenzyldimethylalkylammonium chloride, didecyldimethylammonium chloride, dioctyldimethylammonium chloride, N-hexadecyltrimethylammonium chloride, 1-hexadecylpyridinium chloride, iminoctadine tris (albesilate);

Iodine Derivatives Such As:

diiodomethyl p-tolyl sulfone, 3-iodo-2-propynyl alcohol, 4-chlorophenyl-3-iodopropargylformal, 3-bromo-2,3-diiodo-2-propenyl ethylcarbamate, 2,3,3-triiodoallyl alcohol, 3-bromo-2,3-diiodo-2-propenyl alcohol, 3-iodo-2-propynyl n-butylcarbamate, 3-iodo-2-propynyl n-hexylcarbamate, 3-iodo-2-propynyl-cyclohexyl-carbamate, 3-iodo-2-propynyl phenylcarbamate;

Phenols Such As:

tribromophenol, tetrachlorophenol, 3-methyl-4-chlorophenol, 3,5-dimethyl-4-chlorophenol, dichlorophene, 2-benzyl-4-chlorophenol, triclosan, diclosan, hexachlorophene, p-hydroxybenzoate, o-phenylphenol, m-phenylphenol, p-phenylphenol 4-(2-tert-butyl-4-methylphenoxy)phenol, 4-(2-isopropyl-4-methylphenoxy)phenol, 4-(2,4-dimethylphenoxy)phenol and their alkali metal salts and alkaline earth metal salts;

Microbicides with an Activated Halogen Group Such As:

bronopol, bronidox, 2-bromo-2-nitro-1,3-propanediol, 2-bromo-4′-hydroxyacetophenone, 1-bromo-3-chloro-4,4,5,5-tetramethyl-2-imidazolidinone, β-brom-β-nitrostyrene, chloracetamide, chloramine T, 1,3-dibromo-4,4,5,5-tetramethyl-2-imidazolidinone, dichloramine T, 3,4-dichloro-(3H)-1,2-dithiol-3-one, 2,2-dibromo-3-nitrilepropionamide, 1,2-dibromo-2,4-dicyanobutane, halane, halazone, mucochloric acid, phenyl (2-chlorocyanovinyl) sulfone, phenyl (1,2-dichloro-2-cyanovinyl) sulfone, trichloroisocyanuric acid;

Pyridines Such As:

1-hydroxy-2-pyridinethione (and their Cu, Na, Fe, Mn, Zn salts), tetrachloro-4-methylsulfonylpyridine, pyrimethanol, mepanipyrim, dipyrithion, 1-hydroxy-4-methyl-6-(2,4,4-trimethylpentyl)-2(1H)-pyridine;

Methoxyacrylates or Similar Such As:

azoxystrobin, dimoxystrobin, fluoxastrobin, kresoxim-methyl, metominostrobin, orysastrobin, picoxystrobin, pyraclostrobin, trifloxystrobin, 2,4-dihydro-5-methoxy-2-methyl-4-[2-[[[[1-[3-(trifluoromethyl)phenyl]ethylidene]amino]oxy]-methyl]phenyl]-3H-1,2,4-triazol-3-one (CAS-No. 185336-79-2);

Metal Soaps Such As:

salts of the metals tin, copper and zinc with higher fatty acids, resin acids, naphthenoic acids and phosphoric acid, and as, for example, tin naphthenate, tin octoate, tin 2-ethylhexanoate, tin oleate, tin phosphate, tin benzoate, copper naphthenate, copper octoate, copper 2-ethylhexanoate, copper oleate, copper phosphate, copper benzoate, zinc naphthenate, zinc octoate, zinc 2-ethylhexanoate, zinc oleate, zinc phosphate, zinc benzoate;

Metal Salts Such As:

salts of the metals tin, copper, zinc, and also chromates and dichromates, such as, for example, copper hydroxycarbonate, sodium dichromate, potassium dichromate, potassium chromate, copper sulfate, copper chloride, copper borate, zinc fluorosilicate, copper fluorosilicate;

Oxides Such As:

oxides of the metals tin, copper and zinc, such as, for example, tributyltin oxide, Cu₂O, CuO, ZnO;

Oxidizing Agents Such As:

hydrogen peroxide, peracetic acid, potassium persulfate;

Dithiocarbamates Such As:

cufraneb, ferban, potassium N-hydroxymethyl-N′-methyldithiobarbamate, sodium dimethyldithiocarbamate, potassium dimethyldithiocarbamate, macozeb, maneb, metam, metiram, thiram, zineb, ziram;

Nitriles Such As:

2,4,5,6-tetrachloroisophthalonitrile, disodium cyano-dithioimidocarbamate;

Quinolines Such As:

8-hydroxyquinoline and their copper salts;

Other Fungicides and Bactericides Such As:

bethozaxin, 5-hydroxy-2(5H)-furanone, 4,5-benzodithiazolinone, 4,5-trimethylenedithiazolinone, N-(2-p-chlorobenzoylethyl)-hexaminium chloride, 2-oxo-2-(4-hydroxyphenyl)acetohydroxycinnamoyl chloride, tris-N-(cyclohexyldiazeniumdioxy)-aluminum, N-(cyclo-hexyldiazeniumdioxy)-tributyltin or its potassium salts, bis-N-(cyclohexyldiazeniumdioxy)-copper; iprovalicarb, fenhexamide, spiroxamine, carpropamid, diflumetorin, quinoxyfen, famoxadone, polyoxorim, acibenzolar S-methyl, furametpyr, thifluzamide, methalaxy-M, benthiavalicarb, metrafenon, cyflufenamid, tiadinil, tea tree oil, phenoxyethanol,

Ag, Zn or Cu-containing zeolites alone or incorporated into polymeric materials.

Very especially preferred are mixtures with

azaconazole, bromuconazole, cyproconazole, dichlobutrazol, diniconazole, hexaconazole, metaconazole, penconazole, propiconazole, tebuconazole, dichlofluanid, tolylfluanid, fluorfolpet, methfuroxam, carboxin, benzo[b]thiophene S,S-dioxide cyclohexylcarboxamide, fenpiclonil, 4-(2,2-difluoro-1,3-benzodioxol-4-yl)-1H-pyrrole-3-carbonitrile, butenafine, imazalil, N-methyl-isothiazolin-3-one, 5-chloro-N-methylisothiazolin-3-one, N-octylisothiazolin-3-one, dichloro-N-octylisothiazolinone, mercaptobenthiazole, thiocyanatomethylthiobenzothiazole, benzoisothiazolinone, N-(2-hydroxypropyl)-amino-methanol, benzyl alcohol (hemi)-formal, N-methylolchloroacetamide, N-(2-hydroxypropyl)-amine-methanol, glutaraldehyde, omadine, dimethyl dicarbonate, 2-bromo-2-nitro-1,3-propanediol and/or 3-iodo-2-propinyl n-butylcarbamate, bethoxazin, o-phthaldialdehyde.

Apart from with the abovementioned fungicides and bactericides, mixtures with a good efficacy are, moreover, also prepared with other active compounds:

Insecticides/Acaricides/Nematicides Such As:

abamectin, acephate, acetamiprid, acetoprole, acrinathrin, alanycarb, aldicarb, aldoxycarb, aldrin, allethrin, alpha-cypermethrin amidoflumet, amitraz, avermectin, azadirachtin, azinphos A, azinphos M, azocyclotin,

Bacillus thuringiensis, bartluin, 4-bromo-2(4-chlorophenyl)-1-(ethoxymethyl)-5-(trifluoromethyl)-1H-pyrrole-3-carbonitrile, bendiocarb, benfuracarb, bensultap, betacyfluthrin, bifenthrin, bioresmethrin, bioallethrin, bistrilfluron, bromophos A, bromophos M, bufencarb, buprofezin, butathiophos, butocarboxim, butoxycarboxim,

cadusafos, carbaryl, carbofuran, carbophenothion, carbosulfan, cartap, quinomethionate, cloethocarb, 4-chloro-2-(2-chloro-2-methylpropyl)-5-[(6-iodo-3-pyridinyl)methoxy]-3(2H)-pyridazinone (CAS-RN: 120955-77-3), chlordane, chlorethoxyfos, chlorfenapyr, chlorfenvinphos, chlorfluazuron, chlormephos, N-[(6-chloro-3-pyridinyl)-methyl]-N′-cyano-N-methyl-ethaneimidamide, chlorpicrin, chlorpyrifos A, chlorpyrifos M, cis-resmethrin, clocythrin, clothiazoben cypophenothrin clofentezin, coumaphos, cyanophos, cycloprothrin, cyfluthrin, cyhalothrin, cyhexatin, cypermethrin, cyromazin,

decamethrin, deltamethrin, demeton M, demeton S, demeton-S-methyl, diafenthiuron, dialiphos, diazinon, 1,2-dibenzoyl-1(1,1-dimethyl)-hydrazine, DNOC, dichlofenthion, dichlorvos, dicliphos, dicrotophos, difethialone, diflubenzuron, dimethoate, 3,5-dimethylphenyl methylcarbamate, dimethyl-(phenyl)-silyl-methyl-3-phenoxybenzyl ether, dimethyl-(4-ethoxyphenyl)-silylmethyl-3-phenoxybenzyl ether, dimethylvinphos, dioxathion, disulfoton,

eflusilanate, emamectin, empenthrin, endosulfan, EPN, esfenvalerate, ethiofencarb, ethion, ethofenprox, etrimphos, etoxazole, etobenzanid,

fenamiphos, fenazaquin, fenbutatin oxide, fenfluthrin, fenitrothion, fenobucarb, fenothiocarb, fenoxycarb, fenpropathrin, fenpyrad, fenpyroximat, fensulfothion, fenthion, fenvalerate, fipronil, flonicamid, fluacrypyrim, fluazuron, flucycloxuron, flucythrinate, flufenerim, flufenoxuron, flupyrazofos, flufenzine, flumethrin, flufenprox, fluvalinate, fonophos, formethanate, formothion, fosmethilan fosthiazate, fubfenprox, furathiocarb,

halofenocid, HCH, (CAS RN: 58-89-9), heptenophos, hexaflumuron, hexythiazox, hydramethylnon, hydroprene,

imidacloprid, imiprothrin, indoxycarb, iodfenfos, iprinomectin, iprobenfos, isazophos, isoamidophos, isofenphos, isoprocarb, isoprothiolane, isoxathion, ivermectin, lambda-cyhalothrin, lufenuron,

kadedrin

lambda-cyhalothrin, lufenuron,

malathion, mecarbam, mervinphos, mesulfenphos, metaldehyde, methacrifos, methamidophos, methidathion, methiocarb, methomyl, metalcarb, milbemectin, monocrotophos, moxiectin,

naled, NI 125, nicotine, nitenpyram, noviflumuron

omethoate, oxamyl, oxydemethon M, oxydeprofos,

parathion A, parathion M, penfluron, permethrin, 2-(4-phenoxyphenoxy)-ethyl ethylcarbamate, phenthoate, phorate, phosalon, phosmet, phosphamidon, phoxim, pirimicarb, pirimiphos M, pirimiphos A, prallethrin, profenophos, promecarb, propaphos, propoxur, prothiophos, prothoate, pymetrozin, pyrachlophos, pyridaphenthion, pyresmethrin, pyrethrum, pyridaben, pyridalyl, pyrimidifen, pyriproxifen, pyrithiobac-sodium,

quinalphos,

resmethrin, rotenone,

salithion, sebufos, silafluofen, spinosad, spirodiclofen, spiromesifen, sulfotep, sulprofos,

tau-fluvalinate, taroils, tebufenozide, tebufenpyrad, tebupirimphos, teflubenzuron, tefluthrin, temephos, terbam, terbufos, tetrachlorvinphos, tetramethrin, Tetramethacarb, thiacloprid, thiafenox, thiarnethoxarn, thiapronil, thiodicarb, thiofanox, thiazophos, thiocyclam, thiomethon, thionazin, thuringiensin, tralomethrin, transfluthrin, triarathen, triazophos, triazamate, triazuron trichlorfon, triflumuron, trimethacarb,

vamidothion, xylylcarb, zetamethrin;

Molluscicides:

fentin acetate, metaldehyde, methiocarb, niclosamide;

Herbicides and Algicides

acetochlor, acifluorfen, aclonifen, acrolein, alachlor, alloxydim, ametryn, amidosulfuron, amitrole, ammonium sulfanate, anilofos, asulam, atrazine, azafenidin, aziptrotryne, azimsulfuron,

benazolin, benfluralin, benfuresate, bensulfuron, bensulfide, bentazone, benzofencap, benzthiazuron, bifenox, bispyribac, bispyribac-sodium, borax, bromacil, bromobutide, bromofenoxim, bromoxynil, butachlor, butamifos, butralin, butylate, bialaphos, benzoyl-prop, bromobutide, butroxydim,

carbetamide, carfentrazone-ethyl, carfenstrole, chlomethoxyfen, chloramben, chlorbromuron, chlorflurenol, chloridazon, chlorimuron, chlornitrofen, chloroacetic acid, chloransulam-methyl, cinidon-ethyl, chlorotoluron, chloroxuron, chlorpropham, chlorsulfuron, chlorthal, chlorthiamid, cinmethylin, cinofulsuron, clefoxydim, clethodim, clomazone, chlomeprop, clopyralid, cyanamide, cyanazine, cycloate, cycloxydim, chloroxynil, clodinafop-propargyl, cumyluron, clometoxyfen, cyhalofop, cyhalofop-butyl, clopyrasuluron, cyclosulfamuron,

diclosulam, dichlorprop, dichlorprop-P, diclofop, diethatyl, difenoxuron, difenzoquat, diflufenican, diflufenzopyr, dimefuron, dimepiperate, dimethachlor, dimethipin, dinitramine, dinoseb, dinoseb acetate, dinoterb, diphenamid, dipropetryn, diquat, dithiopyr, diduron, DNOC, DSMA, 2,4-D, daimuron, dalapon, dazomet, 2,4-DB, desmedipham, desmetryn, dicamba, dichlobenil, dimethamid, dithiopyr, dimethametryn,

eglinazine, endothal, EPTC, esprocarb, ethalfluralin, ethidimuron, ethofumesate, ethobenzanid, ethoxyfen, ethametsulfuron, ethoxysulfuron,

fenoxaprop, fenoxaprop-P, fenuron, flamprop, flamprop-M, flazasulfuron, fluazifop, fluazifop-P, fuenachlor, fluchloralin, flufenacet, flurneturon, fluorocglycofen, fluoronitrofen, flupropanate, flurenol, fluridone, flurochloridone, fluroxypyr, fomesafen, fosamine, fosametine, flamprop-isopropyl, flamprop-isopropyl-L, flufenpyr flumiclorac-pentyl, flumipropyn, flumioxzim, flurtamone, flumioxzim, flupyrsulfuiron-methyl, fluthiacet-methyl,

glyphosate, glufosinate-aammonium

haloxyfop, hexazinone,

imazamethabenz, isoproturon, isoxaben, isoxapyrifop, imazapyr, imazaquin, imazethapyr, ioxynil, isopropalin, imazosulfuron, imazomox, isoxaflutole, imazapic, ketospiradox, lactofen, lenacil, linuron,

MCPA, MCPA-hydrazide, MCPA-thioethyl, MCPB, mecoprop, mecoprop-P, mefenacet, mefluidide, mesosulfuron metam, metamifop-metamitron, metazachlor, methabenzthiazuron, methazole, methoroptryne, methyldymron, methyl isothiocyanate, metobromuron, metoxuron, metribuzin, metsulfuron, molinate, manolide, monolinuron, MSMA, metolachlor, metosulam, metobenzuron,

naproanilide, napropamide, naptalam, neburon, nicosulfuron, norflurazon, sodium chlorate,

oxadiazon, oxyfluorfen, oxysulfuron, orbencarb, oryzalin, oxadiargyl,

propyzamide, prosulfocarb, pyrazolate, pyrazolsulfuron, pyrazoxyfen, pyribenzoxim, pyributicarb, pyridate, paraquat, pebulate, pendimethalin, pentachlorophenol, pentoxazone, pentanochlor, petroleum oils, phenmedipham, picloram, piperophos, pretilachlor, primisulfuron, prodiamine, profoxydim, prometryn, propachlor, propanil, propaquizafob, propazine, propham, propisochlor, pyriminobac-methyl, pelargonic acid, pyrithiobac, pyraflufen-ethyl,

quinmerac, quinocloamine, quizalofop, quizalofop-P, quinchlorac,

rimsulfuron

sethoxydim, sifuron, simazine, simetryn, sulfosulfuron, sulfometuron, sulfentrazone, sulcotrione, sulfosate,

tar oils, TCA, TCA-sodium, tebutam, tebuthiuron, terbacil, terbumeton, terbuthylazine, terbutryn, thiazafluoron, thifensulfuron, thiobencarb, thiocarbazil, tralkoxydim, tri-allate, triasulfuron, tribenuron, triclopyr, tridiphane, trietazine, trifluoralin, tycor, thdiazimin, thiazopyr, triflusulfuron,

vemolate.

The weight ratios of the active compounds in these active compound combinations can be varied within relatively wide ranges.

Preferably, the active compound combinations comprise the active compound in an amount of from 0.1 to 99.9%, in particular from 1 to 75%, especially preferably from 5 to 50%, the remainder to 100% being one or more of the co-components mentioned above.

The microbicidal compositions or concentrates used for protecting the industrial materials comprise the active compound or the active compound combination in a concentration of 0.01 and 95% by weight, in particular from 0.1 to 60% by weight.

The use concentrations of the active compounds or active compound combinations to be used depend on the nature and the occurrence of the microorganisms to be controlled and on the composition of the material to be protected. The optimum rate of application can be determined by test series. In general, the use concentrations are in the range from 0.001 to 5% by weight, preferably from 0.05 to 1.5% by weight, based on the material to be protected.

With the active compounds or compositions according to the invention, it is possible to replace, in an advantageous manner, the microbicidal compositions available to date by more effective compositions. They have good stability and, in an advantageous manner, a broad activity spectrum.

The active compounds can be applied as such, in the form of their formulations or in the use forms prepared therefrom, such as ready-to-use solutions, suspensions, wettable powders, pastes, soluble powders, dustable products and granules. Application is carried out in a customary manner, for example by watering, spraying, atomizing, broadcasting, dusting, foaming, spreading, etc.

The examples below are given to illustrate the present invention, without limiting it in any way.

### PREPARATION EXAMPLES

### Example 1

At room temperature, 0.88 g of sodium hydride (60% pure, 0.22 mol) is added to 2.81 g of p-methoxythiophenol (0.02 mol) in DMF (30 ml). After 30 minutes, 3.0 g of starting material (0.2 mol) of the formula (III) where R², R³, R⁵, R⁶, R⁷═H and R⁴=Me and X═Cl were added dropwise, and the reaction mixture was heated at 100° C. for 8 h, quenched with ethanol and concentrated. The residue was purified by column chromatography, giving the thiazine of the formula (I) where R¹=4-methoxyphenyl, R², R³, R⁵, R⁶, R⁷=hydrogen and R⁴=methyl.

Yield: 3.7 g (73% of theory), m.p.=57-59° C.

### Example 2

At room temperature, 3.0 g of thiophenol (0.027 mol) and, after the evolution of gas had ceased, 4.46 g of the starting material (0.027 mol) of the formula (III) where R², R³, R⁶, R⁷═H and R⁴, R⁵=Me and X═Cl were added to 1.20 g sodium hydride (60% pure, 0.03 mol) in DMF (40 ml). The reaction mixture was stirred at 100° C. for 2 h, added to water and extracted with methylene chloride, and the extract was dried and concentrated under reduced pressure. The residue was chromatographed on silica gel, giving the thiazine of the formula (I) where R¹=phenyl, R², R³, R⁶ and R⁷═H and R⁴ and R⁵=methyl.

Yield: 3.5 g (54% of theory), m.p.=53-55° C.

### Example 3

0.2 g of sodium hydride (60% pure, 5 mmol) was added to 0.58 g of p-methylthiophenol (4.59 mmol) in DMF (13 ml), and the mixture was stirred for 30 minutes. 1.0 g of the starting material (4.59 mmol) of the formula (III) where R², R³, R⁵, R⁶, R⁷═H, R⁴=cyclohexyl and X═Cl was added, and the reaction mixture was then stirred for 10 minutes and poured into water. After extraction with methylene chloride, drying and concentration under reduced pressure, the residue that remained was chromatographed, giving the thiazine of the formula (I) where R¹=4-methylphenyl, R²,R³,R⁵ R⁶ and R⁷═H and R⁴=cyclohexyl.

Yield: 0.88 g (63% of theory), R_{f}=0.60 (toluene 10/ethyl acetate 1)

### Example 4

292 mg of the starting material (1.17 mmol) of the formula (III) where R², R⁵, R⁶ and R⁷═H, X═OSO₂CH₃ and R³ and R⁴═—CH₂CH₂CH₂CH₂—, 167 mg of 4-methoxythiophenol (1.17 mmol) and a catalytic amount of N,N-dimethyl-4-aminopyridine were initially charged in DMF (20 ml). The reaction mixture was heated at 100° C., and after 1.5 h 118 mg (1.17 mmol) of triethylamine were added and the mixture was stirred at this temperature for a further 72 h. The reaction mixture was poured into water and extracted with methylene chloride, and the extract was dried. The residue which remained after concentration under reduced pressure was chromatographed in silica gel, giving the thiazine of the formula (I) where R¹=4-methoxyphenyl and R³ and R⁴ —CH₂CH₂CH₂CH₂—and R², R⁵, R⁶ and R⁷═H.

Yield: 0.29 g (yield 44% of theory),

R_{f}=0.40 (toluene 10/ethyl acetate 1).

### Example 5

3.19 g of 4-chloroaniline (0.025 mol) were emulsified in water (41 ml), conc. HCl (6.25 ml) was added and the mixture was cooled to 0° C. At this temperature, a solution of 1.83 g of sodium nitrite (0.0265 mol) in water (125 ml) was added dropwise, and the mixture was then stirred for 1 h, followed by addition of urea until iodine/starch paper remained colorless. Using sodium acetate, the solution was adjusted to a pH of about 4.5. At 0° C., the solution was added dropwise to a solution of 3.67 g of the thione of the formula (V) where R², R³, R⁴, R⁵ and R⁷═H and R⁷=Me (0.025 mol) in acetone (125 ml), water (25 ml) and NaOH (1 g in 10 ml of water). The mixture was stirred at 0° C. for 3 h, poured into water and extracted. The residue which remained after drying was chromatographed on silica gel and then purified by kugelrohr distillation, giving the thiazine of the formula (1) where R¹=4-chlorophenyl and R², R³, R⁴, R⁵ and R⁶═H and R⁷=Me.

Yield: 1.39 g (22% of theory), R_{f}=0.40 (toluene 10/ethyl acetate 1)

The substances listed in Table 1 were prepared analogously to Examples 1 to 5:

TABLE 1
Examples of compounds

| Example | R¹ | R² | R³ | R⁴ | R⁵ | R⁶ | R⁷ | Physical data |
|---|---|---|---|---|---|---|---|---|
| 6 | 4-Cl—C₆H₄ | H | H | Me | H | H | H | m.p. = 43-44° C. |
| 7 | 2-MeO—C₆H₄ | H | H | Me | H | H | H | m.p. = 58.5-59° C. |
| 8 | 2-thienyl | H | H | Me | H | H | H | Rf = 0.44 (tol 10/EA 1) |
| 9 | 3,4-Cl₂—C₆H₃ | H | H | Me | H | H | H | Rf = 0.51 (tol 10/EA 1) |
| 10 | 3-MeO—C₆H₄ | H | H | Me | H | H | H | Rf = 0.32 (tol 10/EA 1) |
| 11 | phenyl | H | H | Me | H | H | H | Rf = 0.38 (tol 10/EA 1 |
| 12 | 3-Cl—C₆H₄ | H | H | Me | H | H | H | Rf = 0.38 (tol 10/EA 1) |
| 13 | 2-Cl—C₆H₄ | H | H | Me | H | H | H | Rf = 0.39 (tol 10/EA 1) |
| 14 | 4-F-C₆H₄ | H | H | Me | H | H | H | m.p. = 42-44° C. |
| 15 | 4-Me—C₆H₄ | H | H | Me | H | H | H | m.p. = 62-63.5° C. |
| 16 | 4-CF₃—C₆H₄ | H | H | Me | H | H | H | Rf = 0.52 (tol 10/EA 1) |
| 17 | 3-naphthyl | H | H | Me | H | H | H | m.p. = 61-62° C. |
| 18 | 2,4-Me₂—C₆H₃ | H | H | Me | H | H | H | Rf = 0.43 (tol 10/EA 1) |
| 19 | 4-Br—C₆H₄ | H | H | Me | H | H | H | Rf = 0.51 (tol 10/EAl) |
| 20 | 2-Me—C₆H₄ | H | H | Me | H | H | H | Rf = 0.38 (tol 10/EA 1) |
| 21 | 3-Me—C₆H₄ | H | H | Me | H | H | H | Rf = 0.39 (tol 10/EA 1) |
| 22 | 4-CMe₃—C₆H₄ | H | H | Me | H | H | H | m.p. = 57-59° C. |
| 23 | 3-F—C₆H₄ | H | H | Me | H | H | H | Rf = 0.56 (tol 10/EA 1) |
| 24 | 3-CF₃—C₆H₄ | H | H | Me | H | H | H | m.p. = 68-69° C. |
| 25 | 4-C₆H₅—C₆H₄ | H | H | Me | H | H | H | m.p. = 71-72° C. |
| 26 | 4-OCF₃—C₆H₄ | H | H | Me | H | H | H | Rf = 0.44 (tol 10/EA 1) |
| 27 | 2-naphtyl | H | H | Me | H | H | H | m.p. = 61-62° C. |
| 28 | 2,4-Cl₂—C₆H₃ | H | H | Me | H | H | H | Rf = 0.28 (tol 10/EA 1) |
| 29 | 3,5-Cl₂—C₆H₃ | H | H | Me | H | H | H | Rf = 0.65 (tol 10/EA 1) |
| 30 | 4-C₆H₅—O—C₆H₄ | H | H | Me | H | H | H | Rf = 0.52 (tol 10/EA 1) |
| 31 | 3-C₆H₅—O—C₆H₄ | H | H | Me | H | H | H | Rf = 0.46 (tol 10/EA 1) |
| 32 | 2,6-Cl₂—C₆H₃ | H | H | Me | H | H | H | Rf = 0.49 (tol 10/EA 1) |
| 33 | 2,5-Cl₂—C₆H₃ | H | H | Me | H | H | H | Rf = 0.54 (tol 10/EA 1) |
| 34 | 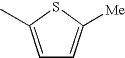 | H | H | Me | H | H | H | Rf = 0.42 (tol 10/EA 1) |
| 35 | 2-thienyl | C₆H₅ | H | H | H | H | H | Rf = 0.21 (tol) |
| 36 | phenyl | C₆H₅ | H | H | H | H | H | Rf = 0.19 (tol) |
| 37 | 4-Me—C₆H₄ | C₆H₅ | H | H | H | H | H | Rf = 0.16 (tol) |
| 38 | 4-MeO—C₆H₄ | C₆H₅ | H | H | H | H | H | m.p. = 127-130 |
| 39 | 4-F—C₆H₄ | C₆H₅ | H | H | H | H | H | Rf = 0.32 (tol) |
| 40 | 4-Cl—C₆H₄ | C₆H₅ | H | H | H | H | H | m.p. = 68-70 |
| 41 | 4-CF₃—C₆H₄ | C₆H₅ | H | H | H | H | H | Rf = 0.47 (tol) |
| 42 | 3-F—C₆H₄ | C₆H₅ | H | H | H | H | H | Rf = 0.31 (tol) |
| 43 | 3-Me—C₆H₄ | C₆H₅ | H | H | H | H | H | Rf =0.25 (tol) |
| 44 | 4-MeO—C₆H₄ | C₆H₅ | H | H | H | H | H | Rf = 0.16 (tol) |
| 45 | 2-thienyl | H | H | Me | Me | H | H | Rf = 0.48 (tol 10/EA 1) |
| 46 | 4-Me—C₆H₄ | H | H | Me | Me | H | H | Rf = 0.54 (tol 10/EA 1) |
| 47 | 4-MeO—C₆H₄ | H | H | Me | Me | H | H | Rf 0.41 (tol 10/EA 1) |
| 48 | 4-Cl—C₆H₄ | H | H | Me | Me | H | H | m.p. = 62-63.5° C. |
| 49 | 4-F—C₆H₄ | H | H | Me | Me | H | H | m.p. = 48-50° C. |
| 50 | 4-CF₃—C₆H₄ | H | H | Me | Me | H | H | Rf = 0.62 (tol 10/EA 1) |
| 51 | 3-F—C₆H₄ | H | H | Me | Me | H | H | Rf = 0.46 (tol 10/EA 1) |
| 52 | 3-Me—C₆H₄ | H | H | Me | Me | H | H | Rf = 0.41 (tol 10/EA 1) |
| 53 | 3-MeO—C₆H₄ | H | H | Me | Me | H | H | Rf = 0.34 (tol 10/EA 1) |
| 54 | 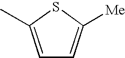 | H | H | Me | Me | H | H | Rf = 0.46 (tol 10/EA 1) |
| 55 | C₆H₅ | Me | Me | H | H | H | H | m.p. = 69-73° C. |
| 56 | 2-thienyl | Me | Me | H | H | H | H | m.p. = 186-188° C. |
| 57 | 3-Me—C₆H₄ | Me | Me | H | H | H | H | Rf =0.18 (tol) |
| 58 | 3-MeO—C₆H₄ | Me | Me | H | H | H | H | Rf = 0.10 (tol) |
| 59 | 4-MeO—C₆H₄ | Me | Me | H | H | H | H | Rf = 0.08 (tol) |
| 60 | 4-Me—C₆H₄ | Me | Me | H | H | H | H | Rf = 0.14 (tol) |
| 61 | 4-Cl—C₆H₄ | Me | Me | H | H | H | H | m.p. = 38-40° C. |
| 62 | C₆H₅ | H | H | H | H | H | Me | Rf = 0.30 (tol 10/EA 1) |
| 63 | 3-MeO—C₆H₄ | H | H | H | H | H | Me | Rf = 0.27 (tol 10/EA 1) |
| 64 | 4-F—C₆H₄ | H | H | H | H | H | Me | Rf = 0.37 (tol 10/EA 1) |
| 65 | 4-Me—C₆H₄ | H | H | H | H | H | Me | Rf = 0.50 (tol 10/EA 1) |
| 66 | 4-MeO—C₆H₄ | H | H | H | H | H | Me | Rf = 0.26 (tol 10/EA 1) |
| 67 | 3-MeO—C₆H₄ | H | H | H | H | H | Me | Rf = 0.37 (tol 10/EA 1) |
| 68 | 4-CF₃—C₆H₄ | H | H | H | H | H | Me | Rf = 0.44 (tol 10/EA 1) |
| 69 | 4-MeO—C₆H₄ | H | H | C₆H₁₁ | H | H | H | Rf = 0.58 (tol 10/EA 1) |
| 70 | 4-Cl—C₆H₄ | H | H | C₆H₁₁ | H | H | H | Rf = 0.64 (tol 10/EA 1) |
| 71 | C₆H₅ | H | H | C₆H₁₁ | H | H | H | Rf ÷ 0.66 (tol 10/EA 1) |
| 72 | 4-F—C₆H₄ | H | H | C₆H₁₁ | H | H | H | Rf = 0.67 (tol 10/EA 1) |
| 73 | 4-CF₃—C₆H₄ | H | H | C₆H₁₁ | H | H | H | Rf = 0.69 (tol 10/EA 1) |
| 74 | 2-thienyl | H | H | C₆H₁₁ | H | H | H | Rf = 0.69 (tol 10/EA 1) |
| 75 | 4-Me—C₆H₄ | H | —CH₂CH₂CH₂— | | H | H | H | Rf = 0.50 (tol 10/EA 1) |
| 76 | 4-Cl—C₆H₄ | H | —CH₂CH₂CH₂— | | H | H | H | Rf = 0.55 (tol 10/EA 1) |
| 77 | 3-Me—C₆H₄ | H | —CH₂CH₂CH₂— | | H | H | H | Rf = 0.50 (tol 10/EA 1) |
| 78 | 3-MeO—C₆H₄ | H | —CH₂CH₂CH₂— | | H | H | H | Rf = 0.38 (tol 10/EA 1) |
| 79 | 2-thienyl | H | —CH₂CH₂CH₂— | | H | H | H | Rf = 0.47 (tol 10/EA 1) |
| 80 | 4-MeO—C₆H₄ | H | —CH₂CH₂CH₂— | | H | H | H | Rf = 0.37 (tol 10/EA 1) |
| 81 | C₆H₅ | H | —CH₂CH₂CH₂— | | H | H | H | Rf = 0.63 (tol 10/EA 1) |
| 82 | 4-Me—C₆H₄ | H | —CH₂CH₂CH₂— | | H | H | H | Rf = 0.56 (to1 10/EA 1) |
| 83 | C₆H₅ | H | —CH₂CH₂CH₂CH₂— | | H | H | H | Rf = 0.55 (tol 10/EA 1) |
| 84 | 3-Me—C₆H₄ | H | —CH₂CH₂CH₂CH₂— | | H | H | H | Rf = 0.47 (tol 10/EA 1) |
| 85 | 2-thienyl | H | —CH₂CH₂CH₂CH₂— | | H | H | H | Rf = 0.45 (tol 10/EA 1) |
| 86 | 3-MeO—C₆H₄ | H | —CH₂CH₂CH₂CH₂— | | H | H | H | Rf = 0.40 (tol 10/EA 1) |
| 87 | 4-Cl—C₆H₄ | H | —CH₂CH₂CH₂CH₂— | | H | H | H | Rf = 0.63 (tol 10/ EA 1) |
| 88 | pentyl | H | H | Me | H | H | H | Rf = 0.67 (tol 10/EA 1) |
| 89 | octyl | H | H | Me | H | H | H | Rf = 0.72 (tol 10/EA 1) |
| 90 | 4-MeO—C₆H₄ | Me | Me | H | H | H | Me | Rf = 0.07 (tol) |
| 91 | 4-Me—C₆H₄ | Me | Me | H | H | H | Me | Rf = 0.12 (tol) |
| 92 | C₆H₅ | Me | Me | H | H | H | Me | Rf = 0.14 (tol) |
| 93 | 4-Cl—C₆H₄ | Me | Me | H | H | H | Me | Rf = 0.19 (tol) |
| 94 | 4-F—C₆H₄ | Me | Me | H | H | H | Me | Rf = 0.17 (tol) |
| 95 | 4-CF₃—C₆H₄ | Me | Me | H | H | H | Me | Rf = 0.14 (tol) |
| 96 | C₆H₅ | Me | H | H | H | H | H | Rf = 0.42 (tol 10/EA 1) |
| 97 | 4-MeO—C₆H₄ | Me | H | H | H | H | H | Rf = 0.86 (tol 10/EA 1) |
| 98 | 4-Me—C₆H₄ | Me | H | H | H | H | Me | Rf = 0.47 (tol 10/EA 1) |
| 99 | 2-thienyl | Me | H | H | H | H | Me | Rf = 0.15 (tol 10/EA 1) |

### Use Example A

To demonstrate the activity against fungi, the minimum inhibitory concentrations (MIC) of agents according to the invention were determined:

In each case, the active compounds according to the invention, in concentrations of from 0.1 mg/l to 5000 mg/l, were added to an agar which had been prepared using malt extracts. After the agar had solidified, it was contaminated with pure cultures of the test organisms listed in Table 3. The MIC was determined after 2 weeks of incubation at 28° C. and 60 to 70% relative atmospheric humidity. The MIC is the lowest concentration of active compound at which there is no colonization by the microbial species used; it is indicated in Table 2.

TABLE 2
Minimum inhibitory concentrations (ppm) of compounds of the
formula (I) according to the invention

| Example | Penicillium | Chaetomium | Aspergillus |
|---|---|---|---|
| No. | brevicaule | globosum | niger |
| 51 | <200 | <200 | 200 |
| 45 | <50 | <50 | <200 |
| 6 | 20 | 20 | 50 |
| 11 | <50 | <50 | <50 |
| 1 | <100 | <100 | <200 |
| 67 | <100 | <100 | <200 |
| 62 | <50 | <100 | <100 |
| 79 | 100 | <100 | |
| 81 | 200 | <100 | |
| 4 | | 100 | |
| 82 | | 100 | |
| 61 | | 200 | |

### Use Example B

To test dispersion coatings for resistance to mold, the following procedure was adopted:

The paint to be tested was applied to both sides of a suitable base. To obtain results which are close to practice, some of the test specimens were rinsed out with running water (24 h, 20° C.) before the test for mold resistance; others were treated with a current of warm fresh air (7 days, 40° C.).

The samples prepared in this way were then placed on an agar nutrient medium, and both samples and nutrient medium were contaminated with fungal spores. After 2-3 weeks storage (29±1° C., 80-90% rel. atmospheric humidity), the samples were compared.

The coating is considered to be permanently mold-resistant if the sample remains free from fungus or at most a slight border infestation can be detected.

For the contamination, fungal spores of the following mold fungi were used, which are known as paint destroyers or are frequently encountered on coatings:
Alternaria tenuis
Aspergillus flavus
Aspergillus niger
Aspergillus ustus
Cindosporum herbarum
Paecilomyces variotii
Penicillium citrium
Aureobasidium pullulans
Stachybotrys chartarum

Coatings according to recipe A are mold resistant (even after rinsing out and wind tunnel exposure) if they contain, for example, 1.5% (based on solids) of the compound of Example 47.

Recipe A: Exterior dispersion paint based on Acroal 290 D (styrene acrylate)

| | Parts by | |
|---|---|---|
| Trade name | weight | Chemical name |
| Bayer Titan RKB2 | 40 | Titanium dioxide |
| Talkum V58 new | 10 | Magnesium silicate, containing |
| | | water |
| Durcal 5 | 45 | Calcite CaCO₃ |
| Walsroder MC 3000 S 2% | 30 | Methylcellulose |
| H₂O | 6.5 | Distilled water |
| Calgon N 10% | 3 | Polyphosphate |
| Pigmentverteiler A 10% | 1 | Polyacrylic acid salt |
| Agitan 281, 1:1 in Texanol | 1 | |
| White spirit | 5 | Mixture of aliph. hydrocarbons |
| Butyl glycol acetate | 1.5 | Butyl glycol acetate |
| Acronal 290 D (binder) | 71 | Polyacrylic acid ester |
| Total | 219 | |

Solids content 135.5 = 61.6%.

1. A compound of the formula (I) 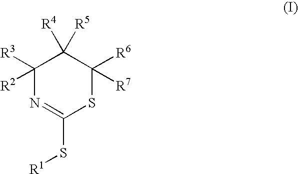
in which
R¹ represents hydrogen or represents in each case optionally substituted alkyl, aryl or heterocyclyl,
and
R² to R⁷ independently of one another represent hydrogen or represent in each case optionally substituted alkyl, aryl or cycloalkyl, where at least one of the substituents R² to R⁷ is different from hydrogen,
or
in each case two substituents from the group consisting of R² to R⁷, together with the C atoms to which they are attached, represent an optionally substituted carbocyclic ring, and the remaining substituents from the group consisting of R² to R⁷ represent hydrogen,
and a metal salt or an acid addition compound thereof.

2. The compound as claimed in claim 1, characterized in that, in formula (I),
R¹ represents hydrogen or represents in each case optionally substituted C₁-C₁₂-alkyl, C₆-C₁₀-aryl or 5- to 7-membered heterocyclyl having 1 to 4 identical or different heteroatoms, which optionally contains a fused-on 6-membered aromatic ring,
and
R² to R⁷ independently of one another represent hydrogen or represent in each case optionally substituted C₁-C₁₂-alkyl, C₆-C₁₀-aryl or C₃-C₈-cycloalkyl, where at least one of the substituents R² to R⁷ is different from hydrogen,
or
two substituents from the group consisting of R² to R⁷, together with the C atoms, to which they are attached, represent an optionally substituted 3- to 12-membered carbocyclic ring and the remaining substituents from the group consisting of R² to R⁷ represent hydrogen.

3. The compound as claimed in at least one of claims 1 or 2, characterized in that, in formula (I),
R¹ represents hydrogen, represents C₁-C₈-alkyl which is optionally mono- to pentasubstituted by identical or different substituents from the group consisting of halogen, C₁-C₆-alkoxy, C₁-C₆-haloalkyl and C₁-C₆-haloalkoxy, represents C₆-C₁₀-aryl which is mono- to trisubstituted by identical or different substituents from the group consisting of halogen, C₁-C₈-alkyl, C1-C6-alkoxy, C₁-C₆-haloalkyl, C₁-C₆-haloalkoxy, hydroxyl, nitro, nitrilo, amino, (di)-C₁-C₆-alkylamino, C₃-C₈-cycloalkyl, phenyl and phenoxy, or represents 5- to 6-membered heterocyclyl having 1 to 3 identical or different heteroatoms from the group consisting of N, O, S, which optionally contains a fused-on aromatic 6-membered ring and which is optionally substituted by C₁-C₈-alkyl,
and
R² to R⁷ independently of one another represent hydrogen, represent C₁-C₁₂-alkyl which is optionally mono- to pentasubstituted by identical or different substituents from the group consisting of halogen, C₁-C₆-alkoxy, C₁-C₆-haloalkyl and C₁-C₆-haloalkoxy, represent C₆-C₁₀-aryl which is optionally mono- to pentasubstituted by identical or different substituents from the group consisting of halogen, C₁-C₈-alkyl, C₁-C₆-alkoxy, C₁-C₆-haloalkyl and C₁-C₆-haloalkoxy, or represent C₃-C₈-cycloalkyl which is optionally mono- to pentasubstituted by identical or different substituents from the group consisting of halogen, C₁-C₆-alkoxy, C₁-C₆-haloalkyl and C₁-C₆-haloalkoxy, where at least one of the substituents R² to R⁷ is different from hydrogen,
or
two substituents from the group consisting of R² to R⁷, together with the C atoms to which they are attached, represent a 3- to 12-membered carbocyclic ring which is optionally mono- to pentasubstituted by identical or different substituents from the group consisting of halogen, C₁-C₈-alkyl, C₁-C₆-alkoxy, C₁-C₆-haloalkyl and C₁-C₆-haloalkoxy, and the remaining substituents from the group consisting of R² to R⁷ represent hydrogen.

4. A process for preparing compounds as claimed in claim 1, characterized in that
mercaptans of the formula (II) or salts thereof 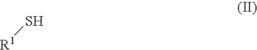
in which
R¹ is as defined in claim 1
are reacted with compounds of the general formula (III) 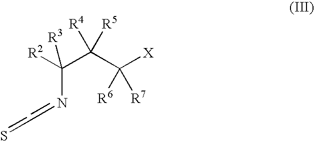
in which
R² to R⁷ are as defined in claim 1,
and represents X halogen or a leaving group,
if appropriate in the present of a diluent and if appropriate in the presence of an acid binder and if appropriate in the presence of a catalyst.

5. A compound of the formula 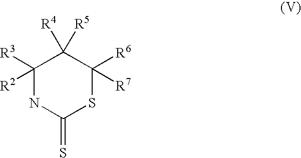
in which
R² to R⁷ are as defined in claim 1,
or a salt thereof,
except for
4-methyl-1,3-thiazinane-2-thione, CAS 5554-49-4;
4,6,6-trimethyl-1,3-thiazinane-2-thione, CAS 6268-74-2;
6-methyl-1,3-thiazinane-2-thione, CAS 13091-77-5;
5-methyl-4-phenyl-1,3-thiazinane-2-thione, CAS 37814-88-3;
4,4,6-trimethyl-1,3-thiazinane-2-thione, CAS 79696-63-2
are novel and also form part of the subject-matter of the present invention.

6. A process for preparing compounds as claimed in claim 5, characterized in that compounds of the formula (VII) or salts thereof 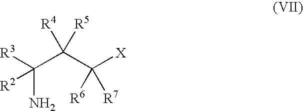
in which
R² to R⁷ are as defined in claim 1,
and
X represents halogen or a leaving group,
are reacted with carbon disulfide, if appropriate in the presence of a diluent and if appropriate in the presence of an acid binder.

7. The use of compounds as claimed in claim 1 as microbicides for protecting industrial materials.

8. The use as claimed in claim 7, characterized in that the industrial materials are wood, plastics, paints, varnishes and plaster.

9. A method for protecting industrial materials against attack and/or destruction by microorganisms, characterized in that at least one compound of the formula (I) as claimed in claim 1 is allowed to act on the microorganism or its habitat.

10. A microbicidal composition for protecting industrial materials, which composition comprises at least one compound of the formula (I) as claimed in claim 1 and at least one solvent or diluent and also, if appropriate, processing auxiliaries and, if appropriate, further antimicrobially active compounds.

11. The composition as claimed in claim 10, characterized in that it comprises at least one further antimicrobially active compound from the group consisting of the fungicides, bactericides, acaricides, nematicides, algaecides and insecticides.

12. An industrial material which comprises at least one compound as claimed in claim 1.

13. The use of a compound as claimed in claim 1 for controlling fungi or for preventing fungal attack.

14. The method for controlling or for preventing fungal attack on plants, characterized in that, in any order or simultaneously, a site of the plant attacked by fungi or threatened by fungal attack or the location in which it grows is treated with at least one compound of the formula (I) as claimed in claim 1.
